(19) United States
(12) Patent Application Publication
(10) Pub. No.: US 2021/0361306 A1
(43) Pub. Date: Nov. 25, 2021

(54) Dormia Basket

(71) Applicant: USHARE Medical Inc., Zhuhai, Guangdong (CN)

(72) Inventors: Aijun Guo, Zhuhai, Guangdong (CN); Chi Weng Lei, Zhuhai, Guangdong (CN)

(21) Appl. No.: 16/320,267

(22) PCT Filed: Feb. 11, 2018
(86) PCT No.: PCT/CN2018/076276

§ 371 Date: Jan. 24, 2019

(30) Foreign Application Priority Data
Sep. 20, 2017 (CN) 201721212156.1

(51) Int. Cl. A61B 17/221

(52) U.S. Cl. CPC A61B 17/221; A61B 2017/00526; A61B 2017/2212

(57) ABSTRACT

The disclosure relates to a dormia basket, including an operation handle, an outer sheath tube, and a basket body. A bottom of the basket body passes through the outer sheath tube and connects to the operation handle. The basket body is formed by winding at least two groups of basket wires. All the basket wires intersect at a top portion of the basket body. Each group of basket wires is formed by at least two metal wires wound around each other. Several slits are formed when two or more metal wires are wound around each other. At least one metal wire in each group of basket wires passes through a slit at a top portion of another group of basket wires, and a metal wire in another group of basket wires passes through a slit of a top portion of each group of basket wires. Compared with other dormia baskets, in the disclosure, the basket body has higher strength, thus improving the safety of surgical operations, provides a large friction force with a calculus, is easier to fixedly capture the calculus, has desirable elasticity and can be unfolded completely inside a body during release.

### FIELD OF THE INVENTION

The disclosure relates to a dormia basket belonging to the technical field of medical instrument.

### BACKGROUND OF THE INVENTION

Dormia baskets are widely used in the treatment of calculuses in various surgical fields such as urology and biliary surgery. Currently, a basket portion of a commercially available tipless dormia basket is usually fixed in a manner of looping and winding. For example, Patent Application No. CN201520186325.3 entitled “Non-steerable Dormia Basket” discloses a dormia basket, of which a basket body is made of at least two basket wires arranged circumferentially, and a top portion of the basket body is formed by looping and winding the middle portions of the basket wires. There are problems that occur easily in the above structure. First, the basket body is formed by fixedly arranging the at least two basket wires, but the basket wires themselves are made of a single metal wire, so the safety is not high. If one of the basket wires is broken, the whole basket body will become loose and scattered, which may bring serious consequences to the calculus removing operation. Second, the single basket wire has a smooth surface, and the friction force for a calculus is insufficient, thus slippage occurs easily during capturing of the calculus, resulting in difficulty in operations. Third, the single basket wire is not sufficiently elastic. When the basket is pushed out from a catheter and released inside a body, it is not easy to completely unfold when encountering a great resistance, which adversely affects the calculus removal operations.

### SUMMARY OF THE INVENTION

To overcome the foregoing deficiencies in the prior art, the disclosure provides a dormia basket which has high safety, provides a large friction force between basket wires and a calculus, thus allowing easy capturing and fixing of the calculus, has desirable elasticity and can be unfolded completely inside a body during release.

The technical solutions of the disclosure for resolving the technical problem are summary as fallows.

A dormia basket includes an operation handle, an outer sheath tube, and a basket body. A bottom of the basket body passes through the outer sheath tube and connects to the operation handle. The basket body is formed by winding at least two groups of basket wires. All the basket wires intersect at a top portion of the basket body. Each group of basket wires is formed by at least two metal wires wound around each other. Several slits are formed when two or more metal wires are wound around each other. At least one metal wire in each group of basket wires passes through a slit at a top portion of another group of basket wires, and a metal wire in another group of basket wires passes through a slit of a top portion of each group of basket wires.

As an improvement to the foregoing technical solution, the bottom of the basket body is formed as a whole by winding and knotting the at least two groups of basket wires.

As an improvement to the foregoing technical solution, each basket wire is formed by winding two metal wires.

As an improvement to the foregoing technical solution, the basket body is formed by winding two groups of basket wires.

The beneficial effects of the disclosure are as follows.

The basket body of the dormia basket is formed by winding at least two groups of basket wires. Each group of basket wires is formed by at least two metal wires wound around each other. Several slits are formed when two or more metal wires are wound around each other, thus the strength of the basket wires and the basket body is increased. When one metal wire is broken, the remaining metal wires still work. When the groups of basket wires are retracted, top portions of the groups of basket wires can be still connected together and do not become loose, and the safety of surgical operations is greatly improved. Friction forces between two or more metal wires and a calculus are increased, and slits formed by the two or more metal wires can easily hold the calculus, making it easier to fixedly capture the calculus and making surgical operations less difficult. In addition, because each group of basket wires is formed by at least two metal wires wound around each other, the elasticity of the basket wires is enhanced, and the basket can be unfolded completely inside a body during release, making calculus removal operations easier in a surgery.

### BRIEF DESCRIPTION OF THE DRAWINGS

The disclosure is further described below with reference to the accompanying drawings and specific embodiments, where:

FIG. 1 is an exploded view according to Embodiment 1 of the disclosure;

FIG. 2 is schematic structural diagram of a basket body according to Embodiment 1 of the disclosure; and

FIG. 3 is a partial enlarged view of an area A of FIG. 2.

### DETAILED DESCRIPTION OF THE EMBODIMENTS

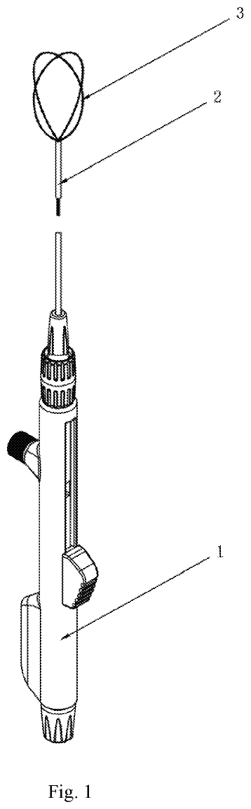

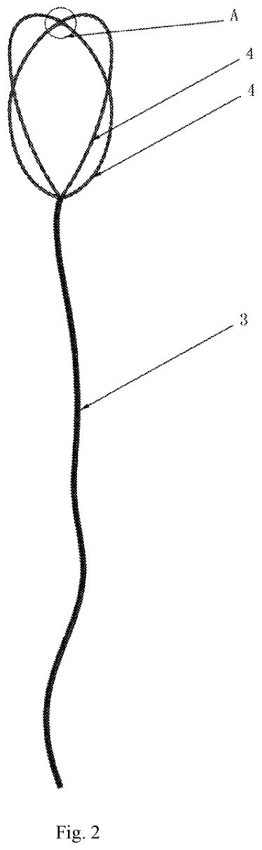

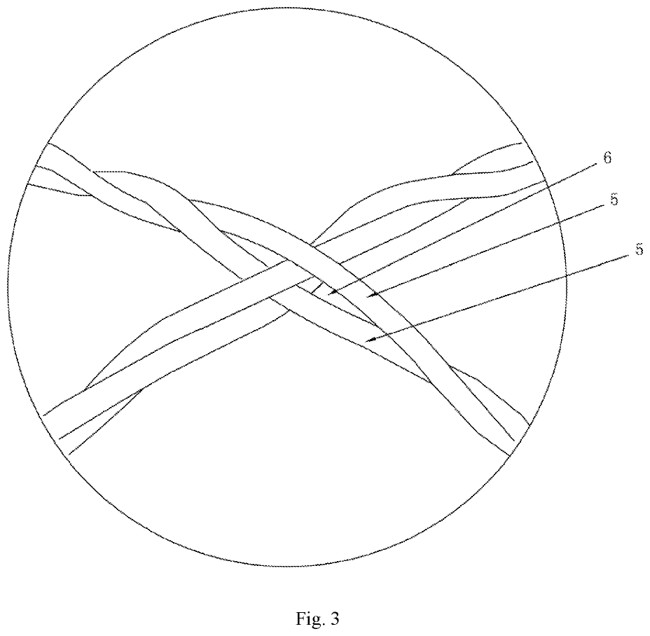

Referring to FIG. 1, FIG. 2, and FIG. 3, a dormia basket includes an operation handle 1, an outer sheath tube 2, and a basket body 3. A bottom of the basket body 3 is connected to the operation handle 1 by passing though the outer sheath tube 2. The basket body 3 is formed by winding at least two groups of basket wires 4. All the basket wires 4 intersect at a top portion of the basket body 3. Each group of basket wires 4 is formed by at least two metal wires 5 wound around each other. Each metal wire can be a stainless steel wire or a nickel-titanium wire or can be made of another memory metal material. Several slits 6 are formed when two or more metal wires 5 wound around each other. At least one metal wire 5 in each group of basket wires 4 passes through a slit 6 at a top portion of another group of basket wires 4, and a metal wire 5 in another group of basket wires 4 passes through a slit 6 at a top portion of each group of basket wires 4.

The bottom of the basket body 3 is formed as a whole by winding and knotting the at least two groups of basket wires 4, so that it becomes smoother for the bottom of the basket body 3 to pass through the outer sheath tube 2.

Referring to FIG. 1, FIG. 2, and FIG. 3, in Embodiment 1, the basket body 3 is formed by winding two groups of basket wires 4. Each basket wire 4 is formed by winding two metal wires 5. One metal wire in one group of basket wires 4 passes through a slit 6 at a top portion of another group of basket wires 4, and one metal wire 5 in another group of basket wires 4 also passes through a slit 6 at a top portion of the group of basket wires 4.

In Embodiment 2 (not shown), there are three groups of basket wires 4. Each basket wire 4 is formed by winding two metal wires 5. The first group and the second group are formed by using the winding in Embodiment 1. One metal wire 5 in the third group of basket wires 4 passes through a slit 6 at a top portion of the first group of basket wires 4 or the second group of basket wires 4, or the two metal wires 5 in the third group of basket wires 4 respectively pass through a slit 6 at a top portion of the first group of basket wires 4 and a slit 6 at a top portion of the second group of basket wires 4, or one metal wire 5 in the third group of basket wires 4 passes through both a slit 6 at a top portion of the first group of basket wires 4 and a slit 6 at a top portion of the second group of basket wires 4.

There can be alternatively four or more groups of basket wires 4. There can be alternatively three or more metal wires 5. At least one metal wire 5 in each group of basket wires 4 passes through a slit 6 at a top portion of another group of basket wires 4, and a metal wire 5 in another group of basket wires 4 passes through a slit 6 at a top portion of each group of basket wires 4. The purpose of arranging a plurality of groups of basket wires 4 is to ensure that the top portions of the groups of basket wires 4 can be still connected together when the groups of basket wires 4 are retracted. The purpose of arranging a plurality of metal wires 5 is to ensure that the basket wires 4 have higher strength. The number of groups of the basket wires 4 and the number of the metal wires 5 can be determined according to the size of a calculus that needs to be captured. When the calculus is smaller, the number of the groups of basket wires 4 is larger and the number of the metal wires 5 is smaller.

The foregoing description merely relates to preferred implementations of the disclosure, and the invention is not limited to the foregoing embodiments. Any embodiment shall fall within the protection scope of the disclosure as long as it achieves the technical effect of the disclosure by the same or similar means.

1-4. (canceled)

5. A dormia basket, comprising:
an operation handle;
an outer sheath tube; and
a basket body with a bottom passing through the outer sheath tube and connecting to the operation handle,
wherein the basket body is formed by winding at least two groups of basket wires,
wherein all the basket wires intersect at a top portion of the basket body,
wherein each group of basket wires is formed by at least two metal wires wound around each other,
wherein several slits are formed when two or more metal wires are wound around each other,
wherein at least one metal wire in each group of basket wires passes through a slit at a top portion of another group of basket wires,
wherein a metal wire in another group of basket wires passes through a slit at a top portion of each group of basket wires.

6. The dormia basket of claim 5, wherein the bottom of the basket body is formed as a whole by winding and knotting the at least two groups of basket wires.

7. The dormia basket of claim 5, wherein each basket wire is formed by winding two metal wires.

8. The dormia basket of claim 5, wherein the basket body is formed by winding two groups of basket wires.
